(19) United States
(12) Patent Application Publication
(10) Pub. No.: US 2010/0189810 A1
(43) Pub. Date: Jul. 29, 2010

(54) Amebacidal ophthalmologic compound

(75) Inventors: Tatsuo Yamamoto, Kasaoka-shi (JP); Yasuo Kurihara, Nagoya-shi (JP); Mutsumi Nakano, Nagoya-shi (JP); Takayoshi Kaizuka, Tokyo (JP)

Correspondence Address: FRISHAUF, HOLTZ, GOODMAN & CHICK, PC, 220 Fifth Avenue, 16TH Floor, NEW YORK, NY, 10001-7708, US

(73) Assignees: Tatsuo Yamamoto, Kasaoka-shi (JP); Mutsumi Nakano, Nagoya-shi (JP); Sinanen Zeomic Co., Ltd., Nagoya-shi (JP); Daisho Sangyo Co., Ltd., Tokyo (JP)

(21) Appl. No.: 12/455,630

(22) Filed: Jun. 4, 2009

(30) Foreign Application Priority Data
Jan. 23, 2009 (JP) 2009-012539

(51) Int. Cl. A61K 33/38; A61P 33/04

(52) U.S. Cl. 424/618

(57) ABSTRACT

An anti-amoebatic ophthalmologic compound which includes an inorganic carrier in which silver ions and hydrogen ions are added. The carrier includes silver ions and hydrogen ions in an amount of more than 1 ppm. The compound can be suspended in an aqueous solution. The aqueous solution may be controlled in a pH value of 5 to 8. The compound may be formed as a molded resinous piece containing an inorganic carrier including silver ions and hydrogen ions in an amount more than 0.01 wt %. The compound is stable, while sustaining a high anti-amoebatic property.

### FIELD OF THE INVENTION

The present invention relates generally to an amebacidal or anti-amoebatic opthalmologic compound for preventing the corneal infection due to amoebida.

### BACKGROUND OF THE INVENTION

In the field of opthalmology, it is well known in the art to deliver medicines such as an antimocrobial agent and/or bacteriocide through dropping as a countermeasure against the maicroorganismal infection.

However, it is reported in many European and American countries from about 1974 that the existent medicines have substantially no effects on the corneal infection due to amoebida such as acanthamoeba or eumycetes.

The cases of the infection due to amoebida have been found among the many contact lens users so that it has been required to make countermeasures against the such infection of the contact lens user soon.

It is strongly desired to establish a significant means for coping with the infection due to acanthamoeba which is the main cause of the especially serious cerotitis, particularly in its cyst condition in which acanthamoeba has made serious affects in combination with the other eumycetes.

However, the amoeba in the cyst condition has high fastness against many medicines including an antimicrobial agent so that there are substantially no effective means thereagainst.

Considering the circumstances mentioned above, medicines effective aganst the amoeba which is the cause of infection are required, and many proposals are made.

For example, proposed are the medicines including chitinase as the effective ingredient thereof (see the related patent 1 cited hereinbelow), the contact lens care compound including hexadecyl-phosphoryl choline as the effective ingredient thereof (see the related patent 2 cited hereinbelow), additives for eye-care products including polyvalent cationic chelete as the effective ingredient thereof (see the related patent 3 cited hereinbelow), and so on.

However, these medicines and therapeutics are very problematic in that the effective ranges of applications of theirs are limited, they are limited in the form of the aqueous solution, and the effective ingredients lost their effectiveness through the hot sterilization process.

In conclusion, there are substantially no countermeasures against amoebida.

[patent 1] Japanese patent laid open public disclosure Hei 04 (1992) -352728

[patent 2] Japanese patent application serial No. 2008-532586

[patent 3] Japanese patent laid open public disclosure 2005-177515

### DISCLOSURE OF THE INVENTION

### Problem or Problems to be Solved by the Invention

The object of the present invention is to provide a opthalmologic compound sustaining highly effective anti-amoebatic property constantly, the compound can also have a form of the aqueous solution and a form of the molded resinous piece.

### The Means for Solving the Problem or Problems

In accordance with the invention, the anti-amoebatic opthalmologic compound includes the inorganic carrier to which silver ions and hydrogen ions are added.

The anti-amoebatic opthalmologic compound further includes the inorganic carrier containing silver ions and hydrogen ions more than 1 ppm, and the carrier is suspended in the aqueous solution controlled in its pH value to 5-8.

The anti-amoebatic opthalmologic compound may be formed as the molded resinous piece including the inorganic carrier containing silver ions and hydrogen ions more than 0.1 wt %.

Effect to be Obtained from the Invention

The opthelmologic compound in the variety of forms such as the aqueous solution or the resinous molded piece will prevent the corneal infection caused by amoebida such as an acanthamoeba.

### THE PREFERRED EMBODIMENT OR EMBODIMENTS OF THE PRESENT INVENTION

The term that the inorganic carrier to which silver ions and hydrogen ions are added means the inorganic carrier including silver ions for providing antimicrobial property and hydrogen ions for sustaining the antimicrobial property of silver ions.

The inorganic carrier can be selected from the grope including zeolite, zirconium phosphate, apatite, silica gel, activated charcoal, glass phosphate, and borosilicate glass.

The most preferred one of the above recited substances are Zeolite because it can hold a large amount of silver ions in more stable form.

The zeolite can be of the natural product such as A type zeolite, X type zeolite, Y type zeolite, T type zeolite, high silica zeolite, natrolite, mordenite, analsime, clinoptirolite, chabazite, and eliomite.

The synthetic zeolite and semi synthetic zeolite can also be adopted.

The method for producing the inorganic carrier including silver ions and hydrogen ions of the present invention will now be described regarding an embodiment employing the zeolite representative of the inorganic carrier.

The inorganic carrier including silver ions and hydrogen ions of the present invention may be produced through the wet process.

In which process, the zeolite was at first dispensed into the preliminary prepared solutions including silver ions to substitute ion-exchangable ions included in the zeolite for silver ions and hydrogen ions.

The process can be carried out continuously under the condition that the temperature of the solution upon introducing the zeolite is around 10-70° C., preferably 40-60° C. for 3-32 hours, more preferably through a batch-process or a column process for 10-24 hours.

The value of pH of the solution including silver ions is suitably to be controlled to be 5-7.

The solutions including silver ions can normally be provided by dissolving silver salt.

For example, the solutions may be produced, without limitation by adding silver nitrate, silver sulfate, silver perchlorate, silver acetate, diammine silver nitrate, and diamine silver sulfate and so on.

The solutions including hydrogen ions can normally be provided an as acid solution.

For example, the solutions may be produced, without limitation by adding carbonic acid, nitric acid, sulfonic acid, perchloric acid, phosphoric acid, acetic acid, propionic acid, oxalic acid and so on.

The amount of silver ions to be included in the zeolite carrier can be controlled by adjusting the silver ion concentration of the solution to be added or by adjusting the throughput thereof.

For example, the silver ion concentration of the zeolite of 0.2 wt. %-50 wt. % can be obtained by making the silver ion concentration of the solution to be 0.04 M/L-1.15 M/L.

The amount of hydrogen ions to be included in the zeolite carrier can be controlled by adjusting the hydrogen ion concentration of the acid solution to be added or by adjusting the throughput thereof.

For example, the hydrogen ion concentration of the zeolite of 0.2 wt. %-3 wt. % can be obtained by making the hydrogen ion concentration of the solution to be 0.06 M/L-0.35 M/L.

The solution including silver ions and hydrogen ions to be reacted by the zeolite can also include other cationic ions.

For example, zinc ion, copper ion, mangan ion, nickel ion, magnesium ion, calcium ion, ammonium ion, and so on can be added for enhancing the stability of the zeolite carrying silver ions.

Such cationic ions can be carried simply by adding to the solution.

In accordance with the present invention, the diameter of the particle including silver ions and hydrogen ions can preferably be 0.5 μm-20 μm.

The inorganic carrier including silver ions and hydrogen ions of the present invention can be used as a variety of opthelmologic compounds such as instillating preparation, contact lens cleaner, disinfectant, preservatives, neutralizer, rinsing solution, moisturizing and lubricating solution, contact lens, and contact lens case and so on.

Such products can be provided as the aqueous solution or the molded resinous piece.

When it is intended to suspend the inorganic carrier including silver ions and hydrogen ions within the aqueous solution, the loading of the inorganic carrier may preferably be more than 1 ppm.

Additionally, it is necessary to make the pH value of the aqueous solution including the inorganic carrier including silver ions and hydrogen ions to be 5-8 for sustaining its antimicrobial property and for keeping the surface of the eyeball from weak acid to the neutral for the safety.

When it is intended to produce the inorganic carrier including silver ions and hydrogen ions as the molded resinous piece, the loading of the inorganic carrier may preferably be more than 0.1 wt. %.

The opthelmologic compounds can be provided the as liquid solution, gel, mousse, aerosol, and so on.

The compounds may of course include buffer, agent for making the isotonic condition, and chelate, and so on.

Concrete examples of the method for providing compounds will now be described as follows.

### <The Method for Providing Zeolite Including Silver Ions and Hydrogen Ions>

The zeolite to be used in this example is commercially available A type zeolite (Na₂O.Al₂O₃.2SiO₂.XH₂O; average particle diameter is 1.5 μm).

The zeolite is dried in the temperature of 110° C. for 5 hours to provide A type zeolite of the moisture content of 20%.

Then water is added to thus obtained A type zeolite 100 g to make slurry of 1 liter.

The HCl solution of 0.2 N of 4000 ml is added thereto and stirred on the ordinary temperature for 5 hours.

Excess amount of HCl will then be removed therefrom by filtration and washing operations, and then dried in the temperature of 110° C. for 5 hours to provide hydrogen ions added A type zeolite.

In order to add silver ions to thus obtained hydrogen ions added A type zeolite, the hydrogen ions added A type zeolite of 100 g is mixed with water to provide slurry of 1 liter, AgNO₃ solution of 0.2 mol/L of 370 ml is added thereto, and thus obtained solution is stirred on 50° C. for 24 hours.

After effecting filtration and washing operations, it is dried on 110° C. for 5 hours to provide hydrogen ions and silver ions added A type zeolite.

### <Amoeba Used for Evaluating the Compounds>

The amoebas listed hereinbelows are used for evaluating the antiamoebatic effect.

| | |
|---|---|
| Acanthamoeba castellanii | [ATCC 30011] |
| Acanthamoeba polyphaga | [ATCC 30461] |

A solution of 100 ml to which added a predetermined amount of the inorganic carrier carrying hydrogen ions and silver ions is prepared.

The amoeba solution of 10 ml including acanthamoeba of cyst type in the number of 100/ml is also prepared.

These solutions are mixed and stirred on 25° C. for 24 hours, and then the inorganic carrier is removed therefrom through centrifugation.

Thereafter, hydrogen peroxide 3% of 5 ml is added thereto and leaves it at rest for 30 minutes, sodium pyruvate is added, and filtered it through a membrane filter to which neutralizing and sterilizing treatments are effected.

The filter is then placed in the proteosepeptone-yeast extract-glucose (PYG) medium and cultured for 2 weeks.

The viability of the acanthamoeba after cultivation is observed through the visual inspection, and the results are listed below.

| | load(ppm) | ATCC30011(alive) | ATCC30461(alive) |
|---|---|---|---|
| 1 | 5 | undetected | undetected |
| 2 | 30 | undetected | undetected |
| 3 | 200 | undetected | undetected |
| 4 | 60 | undetected | undetected |
| 5 | 10 | undetected | undetected |
| 6 | 0 | detected | detected |

### <The Test for Evaluate Anti-Amoebatic Property of the Molded Resinous Piece Including the Inorganic Carrier>

In order to evaluate anti-amoebatic property of the molded resinous piece, two sorts of polyethylene pieces of the thickness 25 μm are prepared.

The one of which is added the inorganic carrier in 20 wt %, and the other of which is added the inorganic carrier in 3 wt %.

The Amoeba solution of 100 ml including acanthamoeba polyphaga of cyst type in the number of 100/mL is also prepared.

Two sorts of polyethylene film are cut to the size of 5 mm×5 mm.

Cut films of 10 g are immersed respectively in the Amoeba solution and stirred on 25 eba solution and stirred on 25° C. for 24 hours, and t for 24 hours, and the films are removed by means of stainless mesh.

Thereafter, 3% the hydrogen peroxide of 5 ml is added thereto and leaves it at rest for 30 minutes, sodium pyruvate is added, and filtered it through a membrane filter to which neutralizing and sterilizing treatments are effected.

The filter is then placed in the proteosepeptone-yeast extract-glucose (PYG) medium and cultured for 2 weeks.

The viability of the acanthamoeba after cultivation is observed through the visual inspection. No acanthamoeba are detected in the either test.

With respect to the zeolite carrying hydrogen ions and silver ions, the safety thereof is confirmed through the chronic toxicity test, the rabbit eye irritation test, the absorbency, distribution, and the eccrisis test, and the other variety of toxicity tests.

In other words, the opthalmologic compounds including zeolite of the type mentioned above have no adverse influence on the human body, i.e. the safety of the compounds is extremely high.

It is to be appreciated that the invention has been described hereinabove with reference to certain examples or embodiments of the invention but that various additions, deletions, alterations and modifications may be made to those examples and embodiments without departing from the intended sprit and scope of the invention.

1. An anti-amoebatic ophthalmologic compound comprising an inorganic carrier to which silver ions and hydrogen ions are added.

2. The anti-amoebatic ophthalmologic compound claimed in claim 1, wherein the inorganic carrier contains silver ions and hydrogen ions in an amount of more than 1 ppm; and the carrier is suspended in an aqueous solution.

3. The anti-amoebatic ophthalmologic compound claimed in claim 2, wherein the aqueous solution is controlled in a pH value of 5 to 8.

4. The anti-amoebatic ophthalmologic compound claimed in claim 1, wherein the compound is formed as a molded resinous piece including the inorganic carrier containing silver ions and hydrogen ions in an amount more than 0.1 wt %.

5. The anti-amoebatic ophthalmologic compound claimed in claim 1 wherein, the inorganic carrier has a silver content within a range from 0.2 wt % to 50 wt %.

6. The anti-amoebatic ophthalmologic compound claimed in claim 1 wherein, the inorganic carrier has a hydrogen content within a range from 0.2 wt % to 3 wt %.

7. The anti-amoebatic ophthalmologic compound claimed in claim 2, wherein the inorganic carrier has a silver content within a range of 0.2 wt % to 50 wt %.

8. The anti-amoebatic ophthalmologic compound claimed in claim 3, wherein the inorganic carrier has a silver content within a range of 0.2 wt % to 50 wt %.

9. The anti-amoebatic ophthalmologic compound claimed in claim 4, wherein the inorganic carrier has a silver content within a range of 0.2 wt % to 50 wt %.

10. The anti-amoebatic ophthalmologic compound claimed in claim 2, wherein the inorganic carrier has a hydrogen content within a range of 0.2 wt % to 3 wt %.

11. The anti-amoebatic ophthalmologic compound claimed in claim 3, wherein the inorganic carrier has a hydrogen content within a range of 0.2 wt % to 3 wt %.

12. The anti-amoebatic ophthalmologic compound claimed in claim 4, wherein the inorganic carrier has a hydrogen content within a range of 0.2 wt % to 3 wt %.

13. The anti-amoebatic ophthalmologic compound claimed in claim 5, wherein the inorganic carrier has a hydrogen content within a range of 0.2 wt % to 3 wt %.

14. The anti-amoebatic ophthalmologic compound claimed in claim 7, wherein the inorganic carrier has a hydrogen content within a range of 0.2 wt % to 3 wt %.

15. The anti-amoebatic ophthalmologic compound claimed in claim 8, wherein the inorganic carrier has a hydrogen content within a range of 0.2 wt % to 3 wt %.

16. The anti-amoebatic ophthalmologic compound claimed in claim 9, wherein the inorganic carrier has a hydrogen content within a range of 0.2 wt % to 3 wt %.
